(19) United States
(12) Patent Application Publication
(10) Pub. No.: US 2005/0125001 A1
(43) Pub. Date: Jun. 9, 2005

(54) Corneal transplant trimming and suturing device

(76) Inventor: Gabriel Danon, Jerusalem (IL)

Correspondence Address: WELSH & FLAXMAN LLC, 2450 CRYSTAL DRIVE, SUITE 112, ARLINGTON, VA, 22202, US

(21) Appl. No.: 10/504,975

(22) PCT Filed:
(86) PCT No.: PCT/IL03/00102

Related U.S. Application Data
(60) Provisional application No. 60/357,106, filed on Feb. 19, 2002.

(51) Int. Cl. A61F 9/00

(52) U.S. Cl. 606/107

(57) ABSTRACT

A device for preparing a corneal transplant for inserting in a recipient eye. A circular knife trims the extracted corneal transplant on the upper face of a radially—grooved cylinder face, and holds it against that upper surface. Surgical needles are inserted inside the radial grooves on the upper face of the cylinder, pointing upwards in the direction of the upper of the cylinder. The transplant is pierced by the needles when the needles are forced upwards by the pushing action of mechanical pushing means. The medical team relocates the trimmed transplant by handling the stitches. The trimmed transplant is preferably removed from the circular knife by the pressure provided to the void volume of the circular knife.

### FIELD OF THE INVENTION

The present invention relates generally to surgical devices. More particularly, the present invention is in the field of corneal transplantation.

### BACKGROUND OF THE INVENTION

Corneal transplantation is performed using corneal tissue extracted from a donor eye, and inserted into a recipient eye after the faulty tissue has been removed from the recipient's eye. In such operations, care has to be taken to fit the graft in place without distorting the graft tissue for several reasons. The first reason is to allow for complete contact of the graft with the supporting tissue of the recipient's eye surrounding the graft. This is better explained in reference to FIG. 1A-1D. A donor eye is shown in FIG. 1A, generally designated as 10. The cut made around the cornea is designated 12. A dashed circle 14 designates the cornea and is remained untouched by the contours of the cut 12. In FIG. 1B the transplant is shown dislodged from the donor's eye. The outline of the cornea 14 is shown for the sake of clarity by a dashed circle. By a next operative act, as shown in FIG. 1C, a circular tissue 16 from within the limits of the cornea, is extracted by a circular blade, the diameter of which matches the dimensions of an incision made in the recipient's eye. In FIG. 1D a recipient's eye 18 is shown, illustrating a circle 20, which marks the circumference of the circular piece of corneal tissue taken out and replaced by the graft 16, the diameter of which is typically slightly larger than the circular incision in the recipient's eye.

### BRIEF DESCRIPTION OF THE DRAWINGS

FIG. 1A is a schematic description of a prior art incision made in a donor's eye, with the actual cut designated, marking the circumference of the transplant intended for extraction.

FIG. 1B is a schematic description of a dislodged donor transplant as in the prior art;

FIG. 1C is a schematic description of a dislodged donor transplant after having been trimmed as in the prior art;

FIG. 1D is a schematic description of a donor eye with a hole cut in its own cornea as a preparation for a grafting operation as in the prior art;

FIG. 2A is schematic description of a cylinder with radial grooves on its upper face used for trimming a transplant in accordance with the present invention;

FIG. 2B is schematic description of a cylinder composed of segments, with radial grooves on its upper face used for trimming a transplant in accordance with the present invention;

FIG. 3A is a schematic description of a cylinder of the invention on which raw transplant tissue is laid;

FIG. 3B is a schematic description of a cylinder of the invention on which raw transplant tissue is laid and on top of which a circular knife is inserted;

FIG. 3C is a schematic description of a cylinder of the invention on which raw transplant tissue has been trimmed by a circular knife;

4

FIG. 4A is a schematic description of a cylinder of the invention, with a surgical needle being directed at a groove;

FIG. 4B is a schematic description of a cylinder of the invention, with a surgical needle being inserted into a groove;

FIG. 5A is a schematic description of a longitudinal section in a cylinder of the device of the invention containing two opposing surgical needles and a fresh transplant tissue inserted on the cylinder;

FIG. 5B is a schematic description of a longitudinal section in a cylinder of the device of the invention showing two opposing surgical needles and a circular knife pressing against the transplant tissue before cutting commences.

FIG. 5C is a schematic description of a longitudinal section in a cylinder of the device of the invention showing two opposing needles set against the transplant within the circular knife;

FIG. 5D is a schematic description of a longitudinal section in a cylinder of the device of the invention showing two opposing surgical needles piercing the transplant tissue;

FIG. 6A is a schematic description a surgical needle being inserted in the push spring notch;

FIG. 6B is a schematic description of a push-spring and associated needle with suture;

FIG. 7A is a schematic cross sectional view in a cylinder of the invention showing needles subtended by push-springs and trimmed transplant on top;

FIG. 7B is a schematic cross sectional view in a cylinder of the invention showing transplant tissue pierced by needles subtended by push-springs;

FIG. 8A is a general schematic view of a piercing device of the invention with push-springs biased away from the cylinder;

FIG. 8B is a general schematic view of a piercing device of the invention with push-springs closed in on the cylinder;

FIG. 9 is a schematic cross sectional view in a cylinder of an embodiment of the invention in which the needles are dislocated by push rods actuated from within the cylinder;

FIG. 10 is a schematic overview of the cylinder of the invention, with pierced transplant and sutures attached to a retention ring;

FIG. 11 is a schematic overview of the detached transplant with sutures attached to a retention ring;

FIG. 12 is a schematic overview of a cylinder of the invention fitted with a needle retaining ring;

FIG. 13 is a schematic illustration of a sealed circular knife in accordance with an embodiment of the invention.

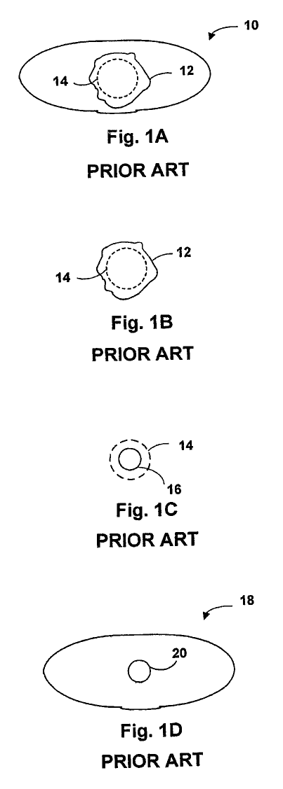

### DETAILED DESCRIPTION OF THE PRESENT INVENTION

In accordance with the present invention, a corneal transplant is extracted from a donor.

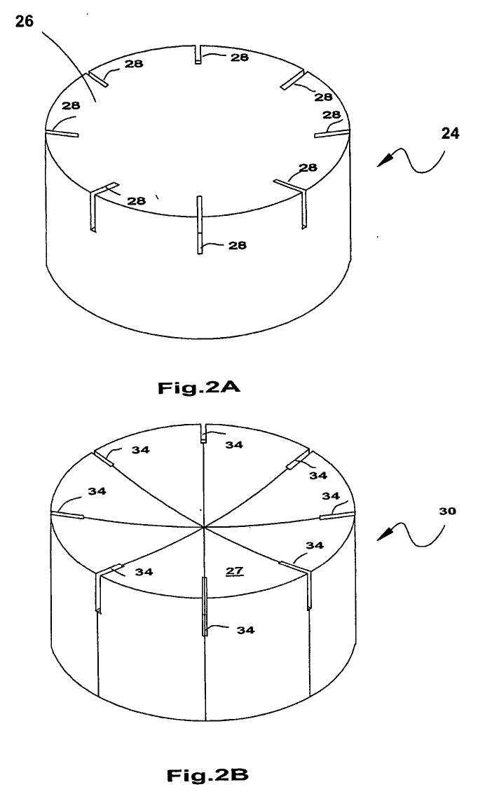

The corneal transplant is set on an upper face of a cutting and piercing device of the invention, to be further trimmed by a circular blade to fit a substantially circular area matching the circular piece removed from the recipient's eye as in the prior art methods. A cutting and piercing device of the invention includes an upright cylinder, the upper part of which containing radial grooves. This can be seen in FIG. 2A to which reference is now made, an isometric view of the cylinder 24 is shown, in which radial grooves 28 are located on the upper face 26 of cylinder 24. Grooves 28 are radially symmetrical, such that each groove has an opposite radial counterpart. In a preferred embodiment of the invention there are 8 grooves. In some embodiments of the invention the cylinder is assembled radially from segments, as can be seen in FIG. 2B to which reference is now made. Thus, cylinder 30 is comprised of segments, the upper face of which jointly forms a cylinder upper face 32, with radial grooves 34 disposed between each segment.

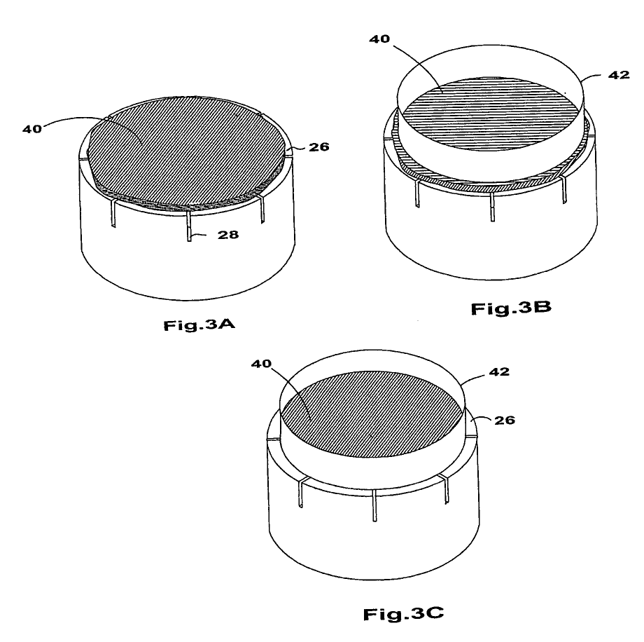

In FIGS. 3A-3C to which reference is now made, the trimming aspect of the invention is described schematically. In FIG. 3A the upper face of the cylinder of the invention is shown covered almost completely by a freshly cut corneal tissue 40. Some of the upper cylinder faces 26 of the cylinder of the invention remains exposed, not covered by the corneal transplant 40. A circular knife is then slid down guided by guiding bars (not shown). In FIG. 3B the circular knife 42 sliding down towards the upper cylinder face is shown just as it meets the transplant 40 on its way down. As the knife is driven down further, it cuts the transplant's tissue, and the parts external to the circular knife are subsequently discarded. The results of this circular trimming are illustrated in FIG. 3C, in which the cut tissue is discarded, and the circular knife 42 contains a circular tissue of the transplant internally. At this stage, the circular knife 42 holds the trimmed transplant tightly. The cutting action may not however prove complete at this stage because the circular knife's drive through the tissue to the bottom is not complete. The circular knife's blade abuts the cylinder's upper face at all the circular knife's circumference except for the grooves. Therefore, it is likely that above the grooves, the transplant's tissue would not be cut completely. In order to solve this problem, after the circular knife 42 has reached the lowest level, i.e. abutting the cylinder, it is turned slightly to the left or to the right, lifted up and again driven down, thereby exerting a final cutting action on the yet uncut tissue. This final cutting action is made possible because of the tight association that the transplant tissue has with the circular knife. Thus, after the first cutting action, when the circular knife is lifted up and turned sideways, the issue moves with it and the uncut strands which were once in line with the groves are now moved to face the hard surface of the upper face.

The Cutting and Piercing Action

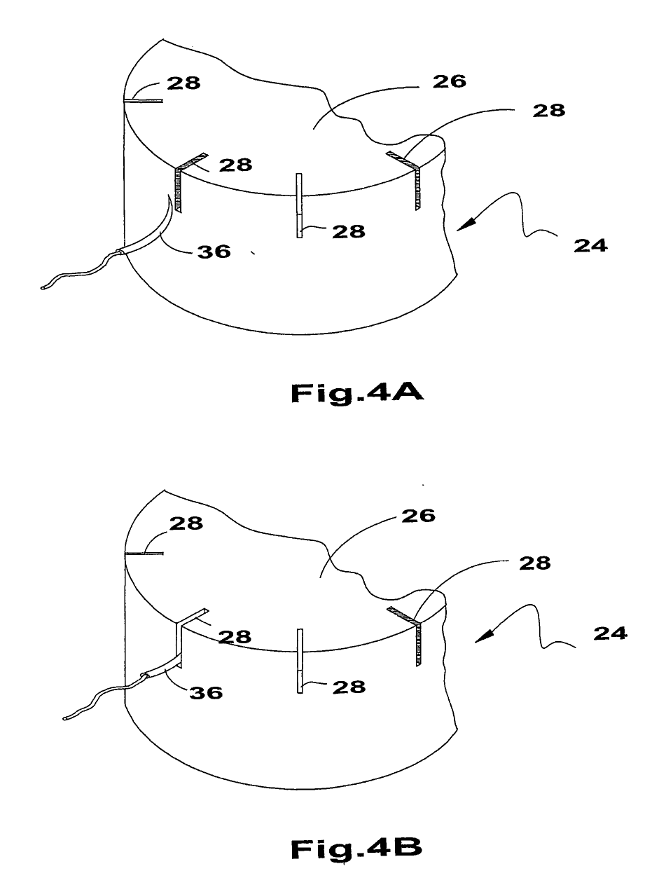

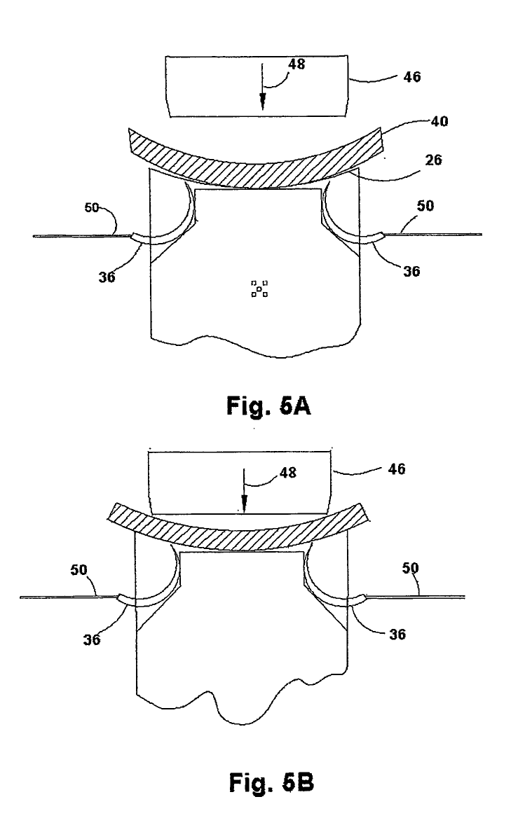

After the corneal transplant is laid on the upper surface of the cylinder, the grooves are located beneath the transplant. However, before the transplant is placed on the upper surface, a surgical needle is inserted inside each groove, respectively. This preparatory action is typically performed in the factory, so that the operating team receives the device of the invention with needles already inserted in the grooves. FIGS. 4A-B to which reference is now made, describe schematically how the surgical needle is inserted in grooves 28 in cylinder 24, in accordance with the device of the present invention. Groove 28 is essentially wide enough and deep enough to accommodate for surgical needle 36. As can be seen in FIG. 4B, the needle is inserted in one of the grooves 28 of cylinder 24 with its pointed side towards the groove, such that the curve of the needle points upwards.

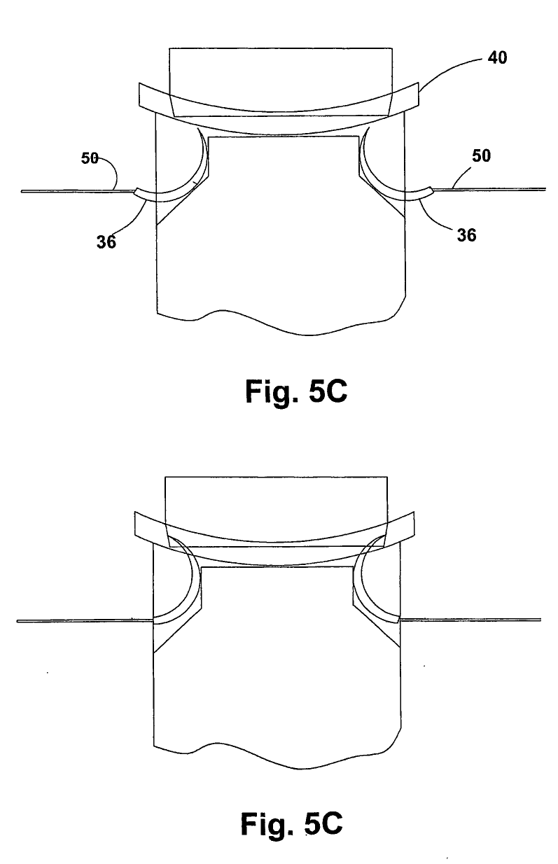

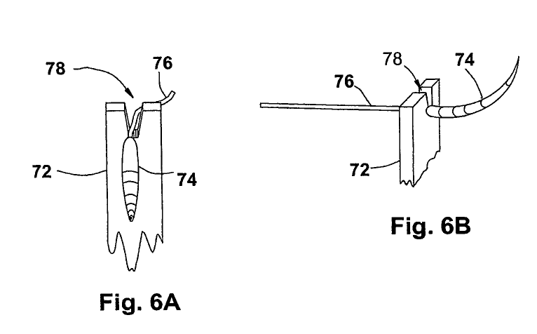

Schematically Illustrated in FIG. 5A to which reference is now made is a cross sectional view in a cutting and piercing device of the invention. In this embodiment of the invention, a transplant, typically freshly cut, is laid on top of the upper cylinder face 26. Transplant 40 is a concave piece of tissue, wherein the convex side is the external side of the eye of the donor. The concave side of the transplant is a very vulnerable part of the cornea and must not be touched. The convex side of the transplant lying on the upper cylinder face corresponds to the outer part of the eye and is not as vulnerable as the other side. Circular knife 46, the elevation of which is shown, begins its cutting action after the transplant has been laid on the upper cylinder face 26, as it moves in the direction of arrow 48. Surgical needles 36 are held in place with their associated stitches 50. In the next stage, which is schematically illustrated in FIG. 5B to which reference is now made, circular knife 46 is pushed in the direction of arrow 48 until it starts cutting the transplant. In FIG. 5C the circular knife is shown pushed to a maximum, such that its blade 52 abuts the upper cylinder face 26. Thereafter, the transplant tissue is trimmed and the cut tissue is discarded as shown in the figure. The Transplant is now held snugly in the circular knife, it can be turned around, raised and pushed again against the upper cylinder face 26, to complete the cutting operation, as explained above. The tips of the surgical needles 36 are shown at a very short distance from the transplant tissue 40. Finally, as described schematically in FIG. 5D the surgical needles 36 and 38 are rotated in the direction of arrows 42 and 44 respectively, such that their tips penetrate the transplant tissue 40. In a preferred embodiment of the invention, the transplant tissue is pierced by the surgical needles, each at its own position in a respective groove. Piercing of the transplant tissue takes place by the forcing action of push-springs. These push-springs are elongated resilient straps having a notch at their upper end for inserting a needle and the attached stitch. The association of a push spring and a surgical needle is better explained with reference to FIGS. 6A-6B. In FIG. 6A, push spring 72 is shown with the needle 74 pointing away from the push spring, and the associated stitch 76 threaded through the notch 78. An isometric side view in FIG. 6B shows the push spring 72 supporting the surgical needle 74 pointing upwards, and the associated stitch 76.

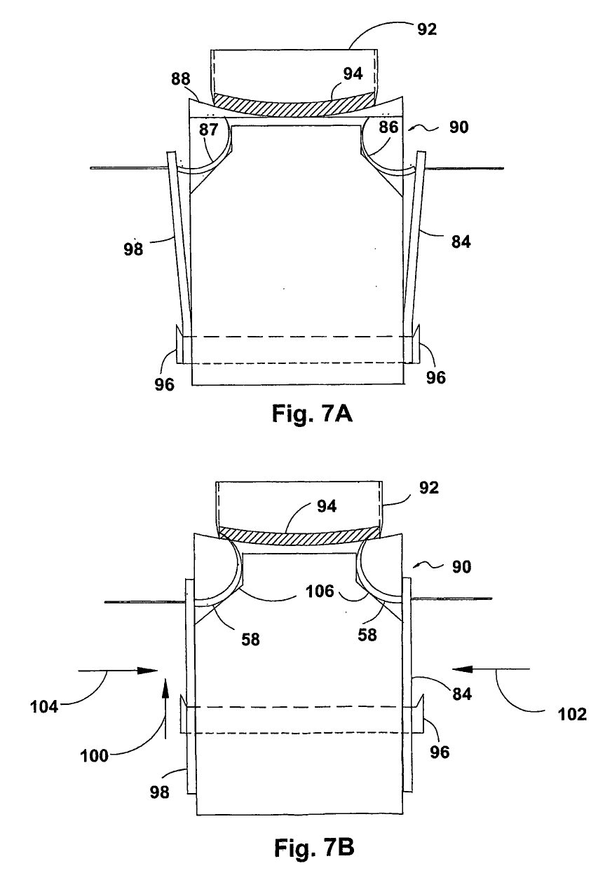

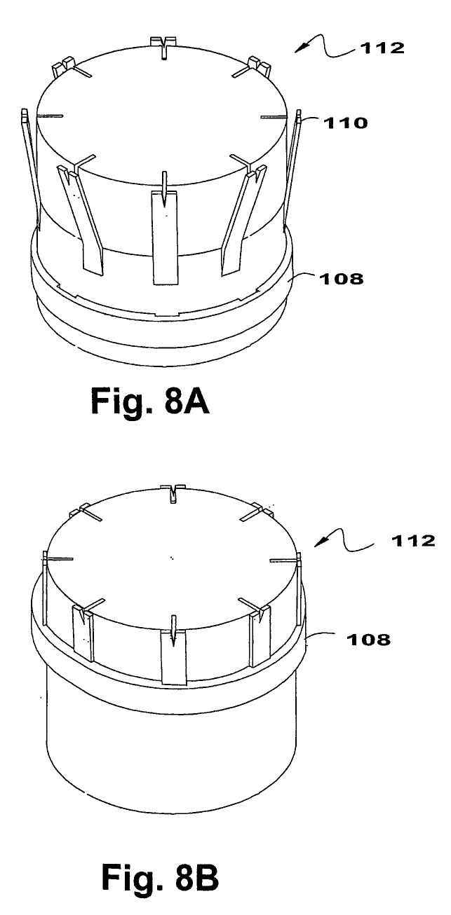

The mechanism which accomplishes the piercing in accordance with the present invention is explained with reference to FIGS. 7A-7B. A cross sectional view of a device of the invention is shown in FIG. 7A, in which a push spring 84 is attached to surgical needles 86 and 87. On the upper face 88 of cylinder 90, circular knife 92 is disposed, containing the trimmed transplant 94. A spring contracting ring (SCR) 96 is disposed at the bottom of push-springs 84 and 98. In FIG. 7B the SCR 96 is shown pushed up in the direction of arrow 100, simultaneously, the push-springs 84 and 98 contract inwards towards the cylinder 90, in the direction of arrows 102 and 104, respectively. This contraction pushes the needles inwards, and in the direction dictated by groove profile 106, a rotational movement is induced which causes the piercing of trimmed transplant 94 by needles 86 and 87 respectively. In FIGS. 8A and 8B to which reference is now made, the movement of the push-springs in association with the movement of the SCR is described. In FIG. 8A is a schematic isometric view of a cylinder of the invention with the push-springs biased away from the cylinder. SCR 108 is shown disposed below the push-springs, and each of the push-springs, such as push spring 108 biased away from the cylinder 112. In FIG. 8B the push-springs are shown contracted, as compelled by the SCR 108 which has moved upwards, abutting cylinder 112.

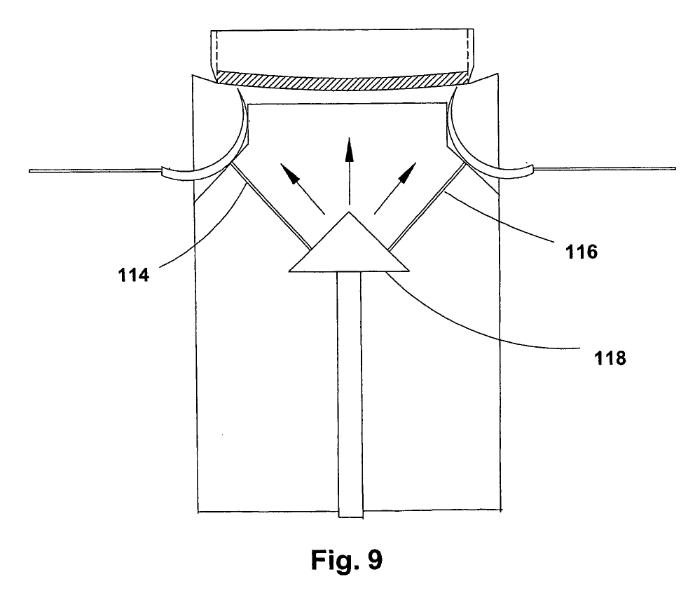

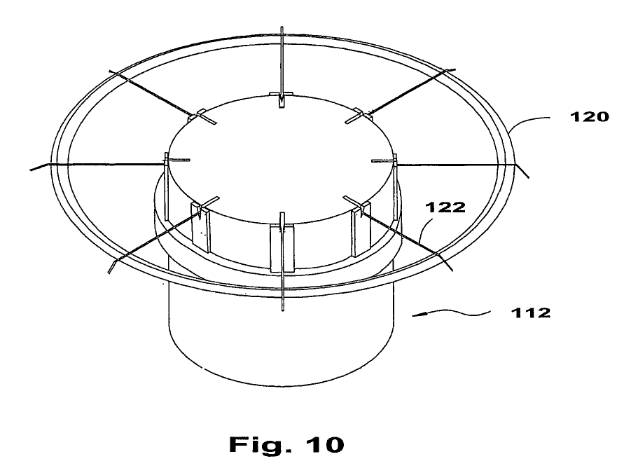

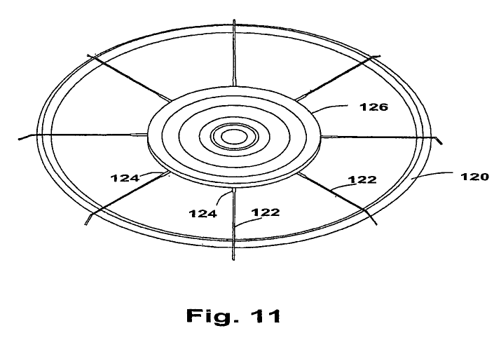

In another embodiment of the present invention, the surgical needles are forced into the transplant tissue each by a push-rod. Accordingly, push-rods 114, 116 are dislocated by a cone 118 as shown in FIG. 9, to which reference is now made. In this embodiment of the invention, the needles are pushed by the push-rods from within the cylinder.

Supporting and Guiding Structures

Once the transplant has been pierced using a device in accordance with the present invention, the transplant can be handed to the surgeon for suturing to the recipient's eye. In accordance with an embodiment of the invention, the transplant is handled with the sutures held by a suture retaining ring (SRR) that keeps the sutures tidy and handy. This is described in FIG. 10 to which reference is now made. SRR 120 retains the sutures, such as suture 122, for example by attaching to a sticky material applied to the SRR or by pressing into grooves carved radially in the ring (not shown). The surgical needles 124 are attached to the transplant 126 underneath. When handled by the medical team, the transplant can be carried about by holding to the SRR. However, since the transplant is held snugly by the circular knife after it has been trimmed, in order to release the transplant from the circular knife, gas pressure is applied to the upper orifice of the circular knife. The internal pressure produced inside the void volume delimited by the circular knife and the transplant increases. Eventually, the transplant is ejected from the circular knife, as illustrated in FIG. 11 to which reference is now made, and is thereafter held by SRR 120, stitches 122 the needles 124.

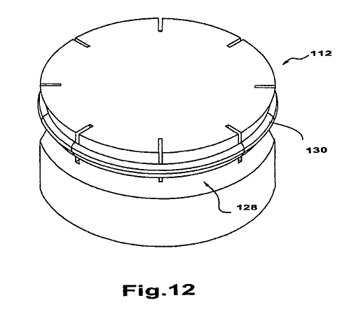

In a preferred embodiment of the invention, the needles are retained in the grooves in the proper direction by a retaining ring as shown in FIG. 12 to which reference is now made. In this embodiment, the envelope of cylinder 112 contains a recess 128 in which a needle retaining ring 130 is disposed. In order to release the pierced transplant, it may be necessary to tear the needle retaining ring.

Handling of the Transplant

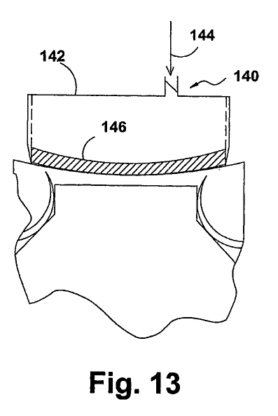

As explained above, the concave side of the transplant is most vulnerable and is not to be touched or otherwise disturbed. In accordance with an embodiment of the invention, the upper opening of the circular knife is sealed off except for an opening as described in FIG. 13 to which reference is now made. A valve, designated 140 is disposed in the upper seal 142 for letting gas in as described by arrow 144, to pressurize the void volume within the closed chamber formed inside the circular knife. Typically, gas used for such a task is air, but other gases can be used such as CO₂ or nitrogen. Subsequently, the transplant tissue 146 is subjected to the pressure formed inside the circular knife. The purpose of this pressurizing is twofold. First, a counter force is provided against the force of the piercing needles. Second, the gas pressure can be used to assist in detaching the tissue from the circular knife. The externally oriented surface of the tissue, facing the inner volume of the circular knife, which is extremely vulnerable, can be kept untouched. When the transplant has been ejected from the circular knife, it can be handed to the medical team. Before grafting, it is turned upside-down, to be further placed over the cut-hole in the recipient's eye. Suturing can commence immediately, using the ready-inserted needles. In practice, in addition to the initial number of needles and sutures supplied, extra suturing is carried out, to enhance the fixation of the transplant to the recipient eye.

1. A cutting and piercing device for preparing corneal transplants for the purpose of grafting in a recipient eye, comprising:
a cylinder for holding on its upper face a raw transplant, wherein said upper face contains radially disposed grooves and wherein each groove has an opposite groove;
a circular knife for trimming a transplant tissue;
one surgical needle disposed in each of said grooves and wherein the tip of each needle points upwards,
means for pushing said needles into said transplant tissue.

2. A cutting and piercing device as in claim 1 and wherein said means for pushing said needles into said transplant tissue are push-springs associated with each needle, and wherein each one of said push-springs has a notch at its upper end for inserting the stitch connected to each one of said needles, respectively.

3. A cutting and piercing device as in claim 1 and wherein said circular knife is sealed off except for an opening for letting gas pressurize the void volume of said knife in order to provide internal pressure in said void volume.

4. A cutting and piercing device as in claim 1 and wherein said surgical needles are retained in the grooves in the proper direction by a retaining ring.

5. A method for preparing an extracted corneal transplant for the purpose of insertion in a recipient eye, comprising the steps of:
trimming said transplant by pushing downwards at least once a circular knife on the upper face of a grooved cylinder;
piercing said trimmed transplant by pushing upwards surgical needles inserted in the grooves of said cylinder, and
releasing said transplant from said circular knife, and further moving it about by holding the stitches of said surgical needles.

6. A method for preparing an extracted corneal transplant for the purpose of insertion in a recipient eye as in claim 5 and wherein said releasing of said transplant is carried out by applying gas pressure to a void volume delimited by said circular knife and said corneal extract.
